(19) United States
(12) Patent Application Publication
(10) Pub. No.: US 2026/0335527 A1
(43) Pub. Date: Oct. 29, 2026

(54) SINGLE-USE ELECTRICALLY-PRESSURIZED IRRIGATION-SUCTION TUBE

(71) Applicant: HAIZE CLINICAL ACHIEVEMENT TRANSLATIONAL MEDICINE RESEARCH INSTITUTE (WUXI) CO., LTD., Wuxi (CN)

(72) Inventors: Jianmin QIAN, Wuxi (CN); Hailong JU, Wuxi (CN); Zengxin QI, Wuxi (CN); Feng ZHU, Wuxi (CN)

(73) Assignee: HAIZE CLINICAL ACHIEVEMENT TRANSLATIONAL MEDICINE RESEARCH INSTITUTE (WUXI) CO., LTD., Wuxi (CN)

(21) Appl. No.: 19/643,941

(22) Filed: Apr. 10, 2026

(30) Foreign Application Priority Data
Apr. 23, 2025 (CN) 2025207773080
Sep. 15, 2025 (CN) 2025219765719
Feb. 9, 2026 (CN) 2026101835843
Feb. 9, 2026 (CN) 2026201912582

(51) Int. Cl. A61M 1/00

(52) U.S. Cl. CPC A61M 1/777; A61M 2205/273; A61M 2205/8206

(57) ABSTRACT

A single-use electrically-pressurized irrigation-suction tube is provided, including handle and irrigation-suction cannula, irrigation-suction cannula being mounted at one end of handle, normal saline connection port and waste liquid and negative pressure connection port being connected to other end of handle, where electric pump and lithium battery for powering electric pump are mounted inside handle, inlet end of electric pump is in communication with normal saline connection port through first irrigation connection tube, outlet end of electric pump is in communication with one port of four-way connector through second irrigation connection tube, remaining three ports are respectively connected to one end of second suction connection tube, one end of irrigation-suction cannula, and one end of anti-clogging irrigation device, other end of second suction connection tube is in communication with waste liquid and negative pressure connection port, flow control valve is mounted on second suction connection tube and/or second irrigation connection tube.

### TECHNICAL FIELD

The present invention relates to a medical device, in particular, to an irrigation-suction tube for irrigating a wound surface in minimally invasive surgery, and specifically, to a single-use electrically-pressurized irrigation-suction tube.

### BACKGROUND

An irrigation-suction tube is a necessary accessory device in minimally invasive surgery, and is for single-use. The irrigation-suction tube is mainly configured to clean a wound surface timely during surgery, to ensure clearness of a surgical field, and timely suck and evacuate waste liquids and residual tissue generated during surgery out of the body, to prevent contamination of the wound surface. Currently, surgical irrigation-suction devices on the market mainly have the following disadvantages: The pressure during irrigation is insufficient, and the pressure is provided mainly by relying on gravity. When the height of a saline bottle for irrigation is insufficient, or the amount of saline in the bottle is insufficient, insufficient irrigation pressure is caused, and the effect of irrigating a wound surface is poor. Sometimes, the healthcare staff even needs to increase the pressure by lifting the saline bottle by hand or using a pressure infusion bag, resulting in very inconvenient use.

In addition, most existing irrigation-suction tubes use a negative pressure source provided by a hospital. A negative pressure flow rate of the negative pressure source is not adjustable. During surgery, the negative pressure may be excessive, causing damage to visceral tissues.

### SUMMARY

An objective of the present invention is to design a single-use electrically-pressurized irrigation-suction tube to resolve problems of insufficient irrigation pressure and inconvenient operation that exist when an existing irrigation-suction tube performs irrigation by relying on gravity.

A first technical solution of the present invention is as follows:

A single-use electrically-pressurized irrigation-suction tube is provided, including a handle 1 and an irrigation-suction cannula 2, the irrigation-suction cannula 2 being fixedly mounted at one end of the handle 1, and a normal saline connection port 3 and a waste liquid and negative pressure connection port 4 being connected to an other end of the handle 1, where an electric pump 5 and a lithium battery 6 for powering the electric pump are mounted inside the handle 1, an inlet end of the electric pump 5 is in communication with the normal saline connection port 3 through a first irrigation connection tube 7, an outlet end of the electric pump 5 is in communication with one port of a four-way connector 15 through a second irrigation connection tube 14, the remaining three ports of the four-way connector 15 are respectively connected to one end of a second suction connection tube 11, one end of the irrigation-suction cannula 2, and one end of an anti-clogging irrigation device 16, an other end of the second suction connection tube 11 is in communication with the waste liquid and negative pressure connection port 4, and a flow control valve 17 is mounted on the second suction connection tube 11 and/or the second irrigation connection tube 14.

The flow control valve 17 includes a valve body 1701 and a valve core 1702, the valve body 1701 is fixed to the handle 1, the valve body 1701 is provided with a liquid outlet 1703 in communication with a valve cavity, the valve core 1702 moves inside the valve cavity of the valve body 1701, and controls a position of the valve core 1702 inside the valve cavity of the valve body 1701 through a limiting pin 1704, to control an opening degree of the liquid outlet 1703 to vary from 0 to a maximum value.

A second technical solution of the present invention is as follows:

A single-use electrically-pressurized irrigation-suction tube is provided, including a handle 1 and an irrigation-suction cannula 2, the irrigation-suction cannula 2 being fixedly mounted at one end of the handle 1, and a normal saline connection port 3 and a waste liquid and negative pressure connection port 4 being connected to an other end of the handle 1, where an electric pump 5 and a lithium battery 6 for powering the electric pump are mounted inside the handle 1, an inlet end of the electric pump 5 is in communication with the normal saline connection port 3 through a first irrigation connection tube 7, an outlet end of the electric pump 5 is in communication with one port of a tee 8 through a connection tube, one end of the tee 8 is in communication with the irrigation-suction cannula 2, and an other end of the tee 8 is in communication with the waste liquid and negative pressure connection port 4 through a first suction connection tube 12.

The tee 8 is mounted inside the handle 1, where one port of the tee 8 is directly and fixedly connected to the irrigation-suction cannula 2.

An irrigation button 9 configured to control starting and stopping of the electric pump and a suction button 10 configured to control flowing of waste liquids are mounted on the handle 1.

Beneficial effects of the present invention are as follows:

In the present invention, pressurization for irrigation is implemented through an electric pump, which can ensure pressure of irrigation and reduce workload of the healthcare staff.

In the present invention, an electric pump and a power supply battery are integrated into a handle, which not only simplifies a structure, but also ensures pressure of irrigation, so that electrical pressurization can ensure normal irrigation, to clarify the surgical field.

The present invention has a simple structure and is convenient to manufacture and use.

The present invention implements an adjustable suction flow rate and/or an adjustable irrigation flow rate. The suction flow rate and/or irrigation flow rate may be controlled through a flow control valve, to avoid possible damage caused by an excessive suction flow rate.

The adjustment is performed in a purely mechanical manner, which is safe, reliable, simple, and convenient.

The present invention implements a back-irrigation function, thereby resolving a problem that an existing irrigation-suction tube cannot perform back-irrigation. In addition, the irrigation structure is simple and convenient, and has adjustable pressure.

The present invention can reduce a flow rate of negative pressure suction from 3000 ml to 250 ml, to ensure surgical safety and avoid negative pressure-caused damage.

### BRIEF DESCRIPTION OF THE DRAWINGS

FIG. 1 is a schematic structural diagram 1 of the present invention;

FIG. 2 is a schematic diagram of a working state when suction is performed through a suction button according to the present invention;

FIG. 3 is a schematic diagram of a state when working is stopped through a suction button according to the present invention;

FIG. 4 is a schematic structural diagram 2 (where internal tubing is omitted) of the present invention;

FIG. 5 is a schematic structural diagram of irrigation tubing of an irrigation-suction tube shown in FIG. 4;

FIG. 6 is a schematic diagram of an irrigation flow direction of an irrigation-suction tube shown in FIG. 4;

FIG. 7 is a schematic structural diagram of suction tubing and flow regulation of an irrigation-suction tube shown in FIG. 4;

FIG. 8 is a schematic diagram of a suction flow direction of an irrigation-suction tube shown in FIG. 4;

FIG. 9 is a schematic structural diagram of anti-clogging irrigation of an irrigation-suction tube shown in FIG. 4;

FIG. 10 is a schematic diagram of a water flow direction of anti-clogging irrigation;

FIG. 11 is a schematic structural cross-sectional view 1 of a flow control valve according to the present invention;

FIG. 12 is a schematic structural cross-sectional view 2 of a flow control valve according to the present invention; and

FIG. 13 is a schematic diagram of Embodiment 2.

### DETAILED DESCRIPTION

The present invention is further described below with reference to the accompanying drawings and embodiments.

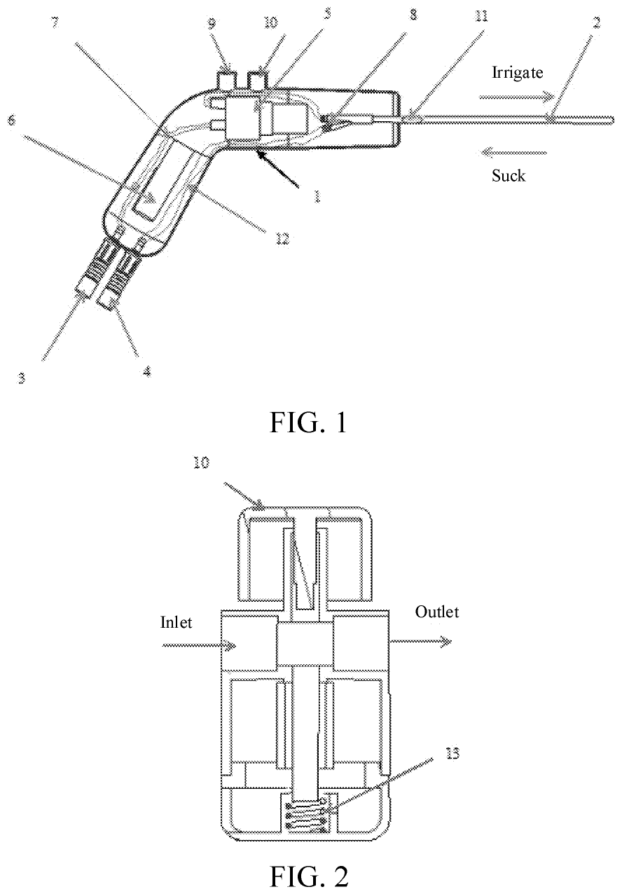

As shown in FIG. 1:

A single-use electrically-pressurized irrigation-suction tube is provided, including a handle 1 and an irrigation-suction cannula 2. The irrigation-suction cannula 2 is fixedly mounted at one end of the handle 1. A normal saline connection port 3 and a waste liquid and negative pressure connection port 4 are connected to an other end of the handle 1. An electric pump 5 (whose model is JT-DC3W-3.7, which is the same below) and a lithium battery 6 for powering the electric pump are mounted inside the handle 1. An inlet end of the electric pump 5 is in communication with the normal saline connection port 3 through a first irrigation connection tube 7. An outlet end of the electric pump 5 is in communication with one port of a tee 8 through a connection tube. A second channel of the tee 8 is in communication with the irrigation-suction cannula 2, and a channel at an other end of the tee 8 is in communication with the waste liquid and negative pressure connection port 4 through a suction connection tube 12. The tee 8 is mounted inside the handle 1, and is directly and fixedly connected to the irrigation-suction cannula 2. An irrigation button 9 configured to control starting and stopping of the electric pump and a suction button 10 configured to control flowing of waste liquids are mounted on the handle 1. As shown in FIG. 1:

Details are as follows:

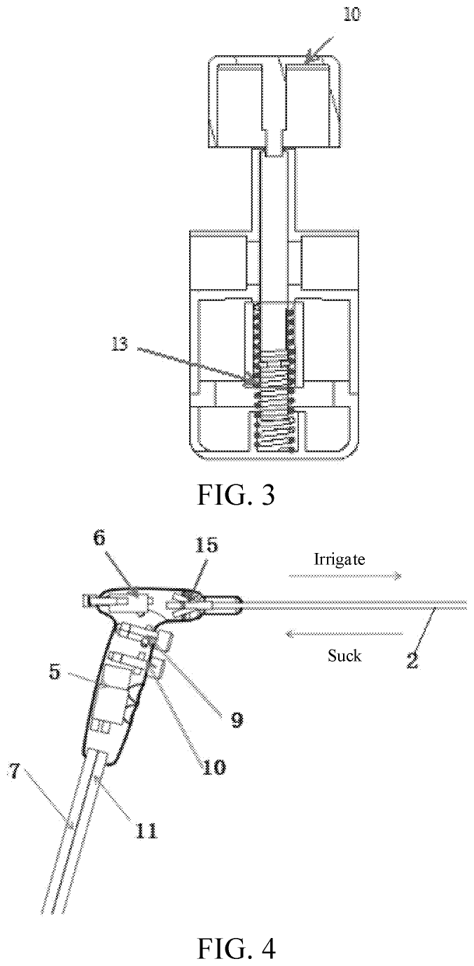

The present invention is configured to irrigate a wound or a wound surface during surgery and suck waste liquids generated during the surgery, and its structure is shown in FIG. 1. The irrigation button 9 is an electrically controlled button, and the suction button 10 is a mechanical button. Irrigation tubing and suction tubing are connected to the irrigation-suction cannula 2 by a tee 8. The irrigation tubing is supplied with normal saline through an electric pump for irrigation, which is powered by a lithium battery, and when the irrigation button is pressed down, the electric pump for irrigation is started. Normal saline in a saline bag is sucked through the tee and output through the irrigation-suction cannula. One end of the suction tubing is connected to a waste liquid bag and a negative pressure source at one end. The negative pressure source provides a power source for sucking waste liquids during surgery. After the suction button is pressed down, the entire suction tubing is connected, and the waste liquids sucked through the irrigation-suction cannula are sucked into the waste liquid bag through the tee and the suction connection tube. After the suction button is released, the entire suction tubing is in a closed state, and there is no negative pressure. After the residue in a filter reaches a specific amount, the residue may be washed away with normal saline. As shown in FIG. 2, after the suction button is pressed down, a liquid channel is fully connected. After the button is released, the button is pushed up due to an elastic force from a spring 13, to close the liquid channel, as shown in FIG. 3.

### Embodiment 2

As shown in FIG. 4 to FIG. 12:

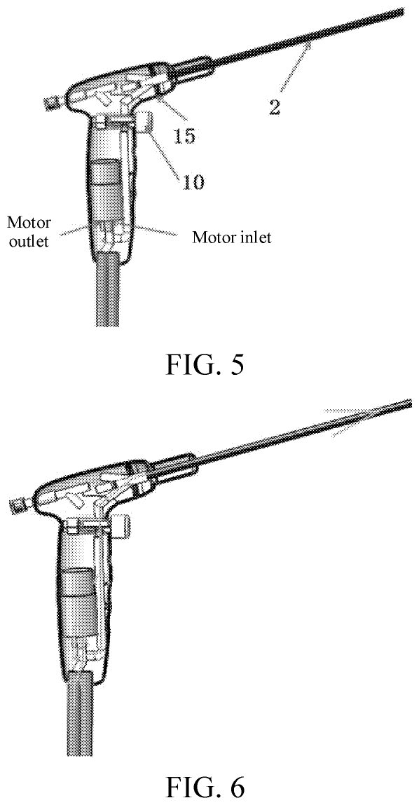

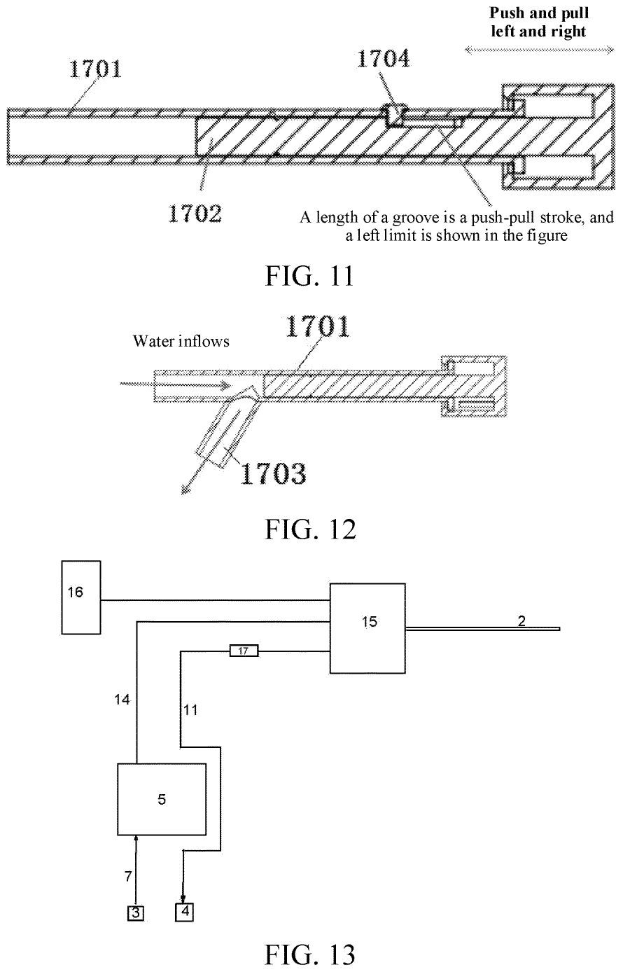

A single-use electrically-pressurized irrigation-suction tube is provided, including a handle 1 and an irrigation-suction cannula 2. The irrigation-suction cannula 2 is fixedly mounted at one end of the handle 1. As shown in FIG. 4, a normal saline connection port 3 and a waste liquid and negative pressure connection port 4 are connected to an other end of the handle 1. An irrigation button 9 (FIG. 4) configured to control starting and stopping of the electric pump and a suction button 10 (FIG. 4) configured to control flowing of waste liquids are mounted on the handle 1. An electric pump 5 and a lithium battery 6 for powering the electric pump are mounted inside the handle 1. An inlet end of the electric pump 5 is in communication with the normal saline connection port 3 through a first irrigation connection tube 7. An outlet end of the electric pump 5 is in communication with one port of a four-way connector 15 through a second irrigation connection tube 14. As shown in FIG. 5, the remaining three ports of the four-way connector 15 are respectively connected to one end of a second suction connection tube 11, one end of the irrigation-suction cannula 2, and one end of an anti-clogging irrigation device 16. An other end of the second suction connection tube 11 is in communication with the waste liquid and negative pressure connection port 4, and a flow control valve 17, as shown in FIG. 11 and FIG. 12, is mounted on the second suction connection tube 11 and/or the second irrigation connection tube 14. Specifically, the flow control valve 17 includes a valve body 1701 and a valve core 1702. The valve body 1701 is fixed to the handle 1. The valve body 1701 is provided with a liquid outlet 1703 in communication with a valve cavity. The valve core 1702 moves inside the valve cavity of the valve body 1701, and controls a position of the valve core 1702 inside the valve cavity of the valve body 1701 through a limiting pin 1704, to control an opening degree of the liquid outlet 1703 to vary from 0 to a maximum value. As shown in FIG. 11, which is a cross-sectional view of limiting at a left limit, the valve core can be pushed and pulled left and right. The valve core is provided with a groove and is limited by a pin shown in the figure. At this moment, the valve is fully open, and a water flow stroke is shown in FIG. 12. The valve core can be pushed and pulled left and right. The valve core is provided with a groove and is limited by a pin. A principle of the flow control valve in the present invention is to change a cross-sectional area of a water channel by pushing and pulling the valve core, to control a flow rate of water. By arranging the flow control valve 17, a flow rate of negative pressure suction can be stably reduced from 3000 ml to a safe value of 250 ml, to ensure surgical safety and avoid negative pressure-caused damage. A flow rate for irrigation can also be adjusted as required.

A working principle of this embodiment is shown in FIG. 13.

Irrigation procedure: As shown in FIG. 5 and FIG. 6, the irrigation button is pressed down, and an electric pump starts to work, to suck saline from the saline bag and discharge the saline from the irrigation-suction cannula. Water flow direction: Normal saline bag-electric pump inlet shown in the figure-electric pump outlet-four-way connector-irrigation-suction cannula.

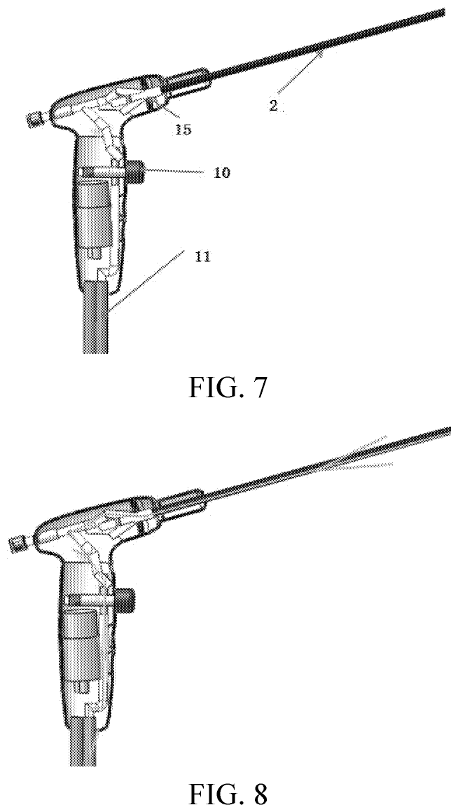

Suction procedure: As shown in FIG. 7 and FIG. 8, the suction button is pressed down, and waste liquids are sucked from the irrigation-suction cannula to the waste liquid bag. Flow direction: Irrigation-suction cannula-four-way connector-flow control valve-waste liquid bag.

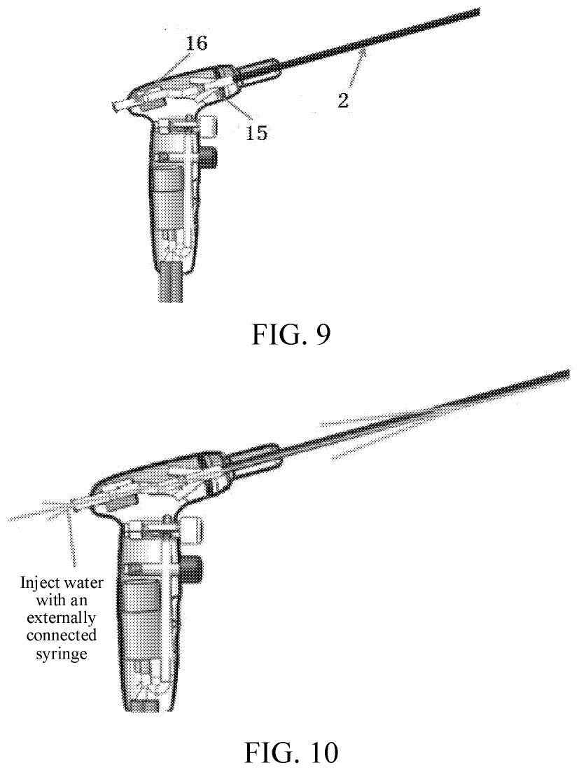

Anti-clogging irrigation procedure: As shown in FIG. 9 and FIG. 10, an anti-clogging irrigation device is externally connected by using a syringe or an item with a similar function, to inject an irrigation liquid, which flows out from the irrigation-suction cannula. Water flow direction: Anti-clogging irrigation device-four-way connector-irrigation-suction cannula.

A principle diagram of a water flow in this embodiment is shown in FIG. 13.

In conclusion, in the present invention, an electric pump built in a handle is used for pressurization, which is significantly different from the existing pressurization using an external moor and an active electric pump, and omits an external power cable, so that an operating environment is optimized. During specific implementation, the flow control valve in the present invention is arranged at an end of a four-way connector connected to the irrigation-suction tube. In this way, the irrigation pressure can also be controlled. Therefore, the flow control valve is the highlight that clearly distinguishes the present invention from the related art. The negative pressure of suction is controllable, which prevents excessive negative pressure from damaging an organ, thereby greatly improving safety of surgery.

All parts not involved in the present invention are the same as the related art or can be implemented by using the related art.

1. A single-use electrically-pressurized irrigation-suction tube, comprising a handle and an irrigation-suction cannula, the irrigation-suction cannula being fixedly mounted at one end of the handle, and a normal saline connection port and a waste liquid and negative pressure connection port being connected to an other end of the handle, wherein an electric pump and a lithium battery for powering the electric pump are mounted inside the handle, an inlet end of the electric pump is in communication with the normal saline connection port through a first irrigation connection tube, an outlet end of the electric pump is in communication with one port of a four-way connector through a second irrigation connection tube, the remaining three ports of the four-way connector are respectively connected to one end of a second suction connection tube, one end of the irrigation-suction cannula, and one end of an anti-clogging irrigation device, an other end of the second suction connection tube is in communication with the waste liquid and negative pressure connection port, and a flow control valve is mounted on the second suction connection tube and/or the second irrigation connection tube.

2. The single-use electrically-pressurized irrigation-suction tube according to claim 1, wherein the flow control valve comprises a valve body and a valve core, the valve body is fixed to the handle, the valve body is provided with a liquid outlet in communication with a valve cavity, the valve core moves inside the valve cavity of the valve body, and controls a position of the valve core inside the valve cavity of the valve body through a limiting pin, to control an opening degree of the liquid outlet to vary from 0 to a maximum value.

3. The irrigation-suction tube according to claim 1, wherein an irrigation button configured to control starting and stopping of the electric pump and a suction button configured to control flowing of waste liquids are mounted on the handle.

4. A single-use electrically-pressurized irrigation-suction tube, comprising a handle and an irrigation-suction cannula, the irrigation-suction cannula being fixedly mounted at one end of the handle, and a normal saline connection port and a waste liquid and negative pressure connection port being connected to an other end of the handle, wherein an electric pump and a lithium battery for powering the electric pump are mounted inside the handle, an inlet end of the electric pump is in communication with the normal saline connection port through a first irrigation connection tube, an outlet end of the electric pump is in communication with one port of a tee through a connection tube, one end of the tee is in communication with the irrigation-suction cannula, and an other end of the tee is in communication with the waste liquid and negative pressure connection port through a first suction connection tube.

5. The irrigation-suction tube according to claim 4, wherein the tee is mounted inside the handle, wherein one port of the tee is directly and fixedly connected to the irrigation-suction cannula.

6. The irrigation-suction tube according to claim 4, wherein an irrigation button configured to control starting and stopping of the electric pump and a suction button configured to control flowing of waste liquids are mounted on the handle.
